(19) United States
(12) Patent Application Publication
(10) Pub. No.: US 2026/0000390 A1
(43) Pub. Date: Jan. 1, 2026

(54) SYSTEMS AND METHODS FOR CLOSURE OF AN ARTERIOTOMY

(71) Applicant: Boston Scientific Scimed, Inc., Maple Grove, MN (US)

(72) Inventors: Blaine Schneider, Corcoran, MN (US); Karl Halden, Sunnyvale, CA (US); Kristen Lipschultz, Minneapolis, MN (US); Sunil Shah, Freemont, CA (US); Brad Steele, Plymouth, MN (US); Zack Tegels, Otsego, MN (US); Bill Whealon, Sunnyvale, CA (US)

(73) Assignee: Boston Scientific Scimed, Inc., Maple Grove, MN (US)

(21) Appl. No.: 19/254,463

(22) Filed: Jun. 30, 2025

Related U.S. Application Data
(60) Provisional application No. 63/666,532, filed on Jul. 1, 2024.

(51) Int. Cl. A61B 17/00

(52) U.S. Cl. CPC A61B 17/0057; A61B 2017/00004; A61B 2017/00477; A61B 2017/00588; A61B 2017/00606; A61B 2017/00615; A61B 2017/00619

(57) ABSTRACT

Arterial closure devices as well as methods for making using arterial closure devices are disclosed. An example arterial closure device may include a proximal gasket configured to be positioned on a first side of a blood vessel wall. The arterial closure device may include a distal footplate configured to be positioned on a second side of the blood vessel wall opposite the proximal gasket. The arterial closure device may include a seal configured to be positioned on the first side of the blood vessel wall between the proximal gasket and the blood vessel wall. A tether may be coupled to at least the proximal gasket and the distal footplate. A securement element may be disposed on the tether. The securement element may be configured to secure the proximal gasket and the seal against the first side of the blood vessel wall.

### CROSS-REFERENCE TO RELATED APPLICATIONS

This application claims the benefit of priority under 35 U.S.C. § 119 of U.S. Provisional Application No. 63/666,532, filed Jul. 1, 2024, the entire disclosure of which is hereby incorporated by reference.

### BACKGROUND

The present disclosure relates generally to medical methods and devices. More particularly, the present disclosure relates to methods and devices for closure of an opening or cut of a blood vessel wall, such as an arteriotomy.

### BRIEF DESCRIPTION OF THE DRAWINGS

FIG. 1 shows a schematic side view of an example gasket style vascular closure device.

FIG. 2 shows a perspective view of an example vascular closure device

FIG. 3 shows a top view of an example footplate.

FIG. 4 shows an example footplate.

FIG. 5 shows a side, schematic view of an example vascular closure device.

FIGS. 6, 7 and 8 show views of examples of the gasket.

FIGS. 9 and 10 show views of examples of the gasket.

FIG. 11 shows an example gasket.

FIG. 12 shows a perspective view of an example delivery device.

FIG. 13 shows a top view of an example delivery device.

FIG. 14 shows a side view of an example delivery device.

FIG. 15 shows a plan view of an example delivery device.

FIG. 16 shows an example delivery device.

FIGS. 17-24 depict a method of use of an example delivery device.

FIG. 25 shows an example delivery device.

FIG. 26A shows a schematic representation of a spring-loaded tether holder assembly coupled to the handle.

FIGS. 26B and 26C show an example of the tether holder assembly that is positioned inside the handle.

FIG. 27 shows a schematic representation of another example of the delivery device.

FIGS. 28A and 28B illustrate a use of an example delivery device.

FIG. 29 illustrates an example vascular closure device.

FIG. 30 illustrates an example vascular closure device.

FIG. 31 illustrates an example vascular closure device.

FIG. 32 illustrates an example vascular closure device.

FIG. 33 illustrates an example vascular closure device.

FIG. 34 illustrates an example vascular closure device.

FIG. 35 illustrates an example vascular closure device.

FIG. 36 illustrates an example method.

FIG. 37 shows an example occluder style closure device.

FIG. 38 shows another example closure device.

FIG. 39 shows a schematic representation of the vasculature in the neck and skull region.

FIG. 40 depicts a closure device positioned at an arteriotomy.

### DETAILED DESCRIPTION

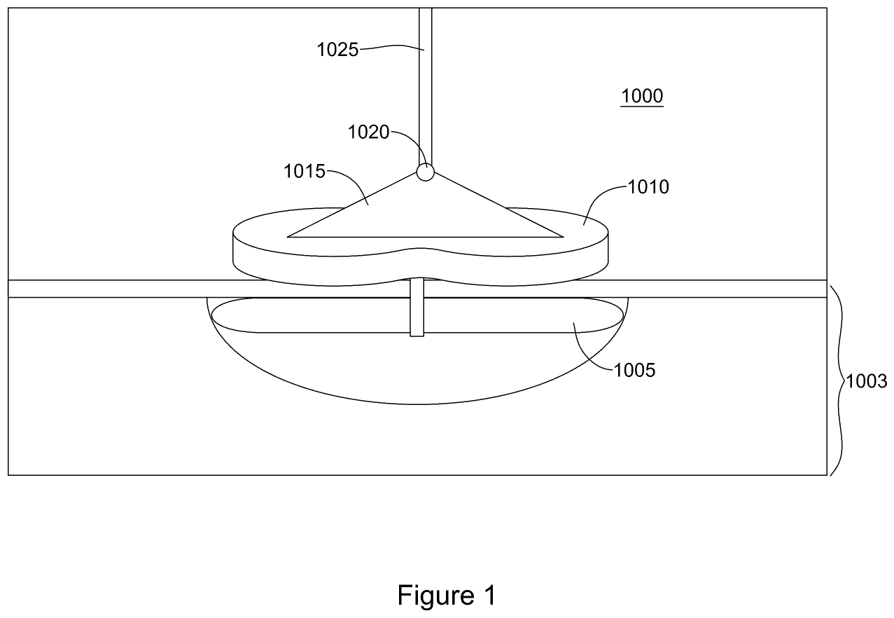

FIG. 1 shows a schematic side view of a gasket style vascular closure device 1000 that is configured to close, seal or inhibit blood leakage of an arteriotomy of a blood vessel 1003. The closure device includes an intravascular footplate 1005, which can serve as a distal seal relative to adjacent tissue, and an extravascular seal 1010, which can serve as a proximal seal relative to adjacent tissue. A blood vessel wall and/or an arteriotomy are positioned between the intravascular footplate 1005 and the extravascular seal 1010. The intravascular footplate 1005 and the extravascular seal 1010 can be bioresorbable. A proximal or upper gasket 1015 is positioned adjacent the extravascular seal 1010 and secured thereto with a securement element 1020 attached to a tether 1025. The tether 1025 is also in contact with the upper gasket 1015, the extravascular seal 1010 and/or the footplate 1005. In use, the vascular closure device 1000 is positioned such that a blood vessel wall in region of the arteriotomy is sandwiched between the extravascular seal 1010 and the intravascular footplate 1005.

The tether 1025 may be a bioabsorbable material. It is configured to prevent or inhibit embolization and to maintain control of the deployment location of the vascular closure device 1000. It extends through the arterial wall tissue and is configured to prevent embolization. In an embodiment, the tether is braided 2-0 PLGA and is attached to the intravascular footplate, although the material of the tether can vary.

The upper gasket 1015 provides pressure (such as a pinching pressure) onto extravascular seal 1010 and/or the footplate 1005. The upper gasket 1015 may be made of a rigid material that can compress the extravascular seal 1010. Or the upper gasket 1015 can be flexible. The upper gasket 1015 can also be bioresorbable.

The extravascular seal 1010 is formed of a material configured to conform to a blood vessel exterior wall in a sealing manner. The extravascular seal 1010 can eliminate or reduce a rate of blood leak through the arteriotomy. It promotes coagulation and hemostasis and can be bioresorbable. In some embodiments, the upper gasket 1015 and the extravascular seal 1010 may be attached to one another and combined into a single component that performs both functions.

The footplate 1005 serves as an anchor to secure the vascular closure device 1000 in place relative to the artery. The footplate 1005 has a minimal side profile. It can be atraumatically shaped from a bioresorbable material. The footplate 1005 can be made of more than one component including a stiffer portion, such as a core, surrounded by one or more flexible wings or a circumferential, flexible flange, as described more fully below.

The securement element 1020 maintains the pinching or compressive force of the upper gasket 1015 onto the extravascular seal 1010 and/or the footplate 1005. The sealing material is configured to conform to the shape of the blood vessel exterior wall. It can be a soft, bioresorbable material.

The components of the vascular closure device 1000 can bioabsorb in a predetermined order. In an embodiment, the footplate absorbs or is endothelialized in approximately 30 days while the remaining components absorb thereafter.

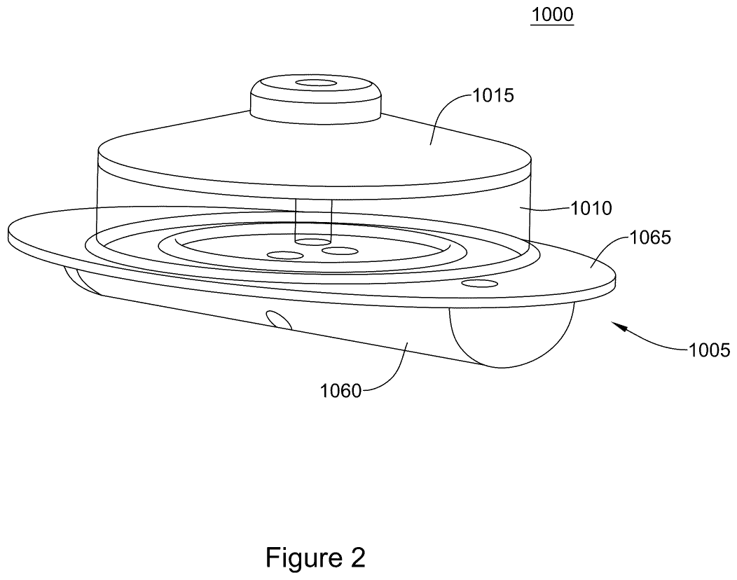

FIG. 2 shows a perspective view of an example vascular closure device 1000. The tether 1025 is not shown in FIG. 2. The gasket 1015 is positioned adjacent the extravascular seal 1010 (not shown). The footplate 1005 is formed of an elongated core 1060 that is surrounded by or adjacent to at least one wing, flange or membrane 1065.

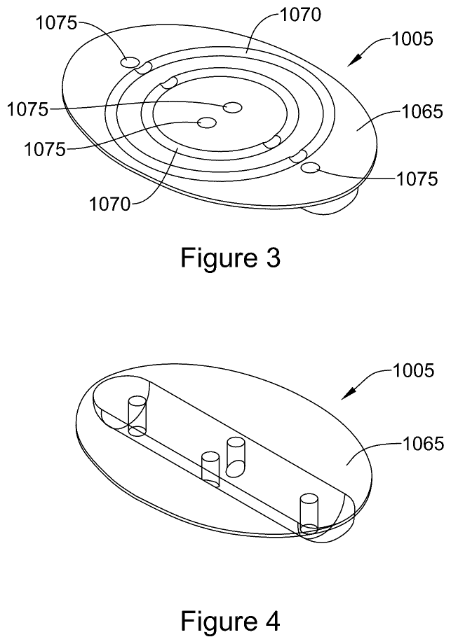

FIG. 3 shows a top view of the footplate 1005. The membrane 1065 can vary in shape and can be for example, circular, ovoid, elliptical, irregular or any other shape. One or more raised surfaces 1070 are located on the footplate 1005. The raised surfaces 1070 are configured to form a sealing relationship with the inner arterial wall. In the illustrated embodiment, the raised surfaces 1070 form concentric rings although the shape can vary. One or more slots 1071 can extend through the raised surfaces 1070 for receiving therein a portion of the tether 1025. FIG. 4 shows the footplate 1005 wherein the membrane does not have the raised surfaces.

The footplate 1005 includes one or more suture or tether holes 1075 sized and shaped to receive the tether 1025 to secure the tether 1025 to the footplate 1005. The tether holes 1075 can be arranged in a variety of patterns. In the illustrated embodiment, the footplate 1005 includes four suture holes 1075 arranged in a diamond pattern wherein the diamond has a long axis that aligns with a long axis of the elongated core 1060. The diamond pattern distributes tether tensile load to minimize buckling. The central holes 1075 are aligned with transverse arteriotomy when the footplate is deployed.

The footplate membrane 1065 is flexible so that it can be folded or otherwise shape manipulated into a delivery shape to fit within a delivery sheath. The material of the footplate membrane is configured so that it resists plastic deformation when deployed in the delivery shape such that it recovers its original shape upon deployment to achieve vessel apposition. The material of the footplate is sufficiently strong to achieve good suture retention and break strength to prevent embolic risk.

In an embodiment, the footplate 1005 is longer than it is wide such as in the region of the core 1060. This promotes self-orientation of the footplate along a blood vessel axis in smaller diameter vessels and also acts as a toggle to resist pulling out of the arteriotomy. In an embodiment, the core is 10 mm long by 7 mm wide although the size can vary. In another example embodiment, the core is 4-8 mm long and 3-8 mm wide. The core 1060 is thick relative to the membrane 1065. The thick core provides strength and stability to the footplate while the membrane provides sealing and atraumatic conformability.

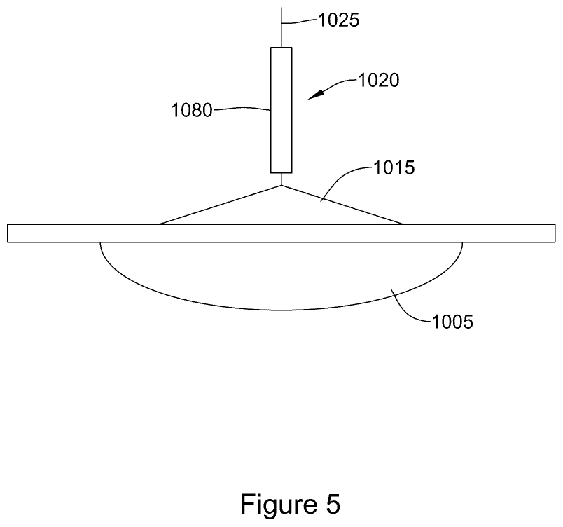

FIG. 5 shows a side, schematic view of an embodiment of the vascular closure device 1000. The securement element 1020 is formed by a thicker or enlarged region 1080 of material that transitions to a smaller region just immediate the gasket 1015. The gasket 1015 can be moved downward along the tether 1025 along the enlarged region 1080 wherein a strong friction is achieved between the enlarged region 1080 and the gasket 1015. Once the gasket 1015 moves past the enlarged region 1080 as it moves downward along the tether, the gasket can pop into place along a thinner, smaller region adjacent the arterial wall. The enlarged region 1080 of the tether acts as an abutment, with thinner, smaller region of achieving a controlled position of the gasket 1015. The enlarged region can also be tapered at one or both ends, or persist to the footplate.

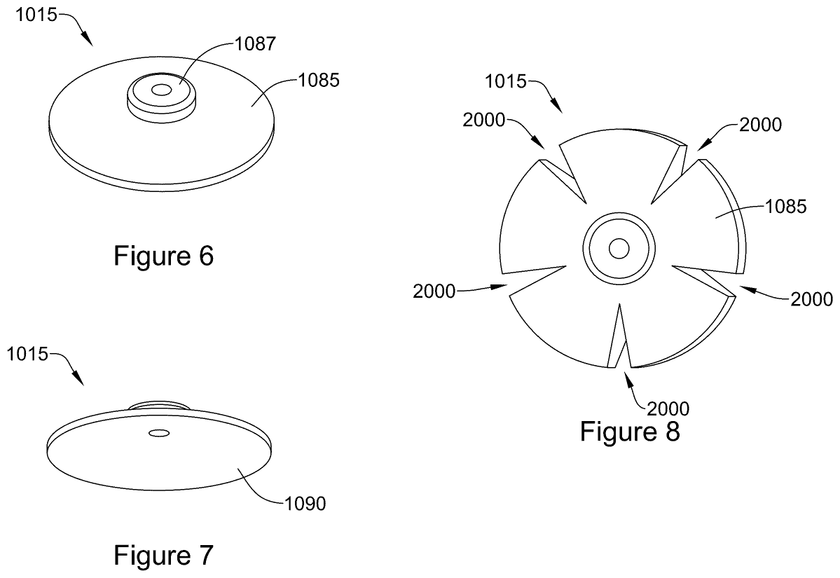

FIGS. 6, 7 and 8 show views of embodiments of the gasket 1015. The embodiment of FIGS. 6 and 7 is generally circular although the shape can vary such as oval, polygon, flat or conical. The gasket 1015 has an upper raised surface 1087 surrounded by a ramped top surface 1085 and a concave bottom surface 1090. In another embodiment shown in FIG. 8, one or more radially extending slits 2000 are positioned on the gasket 1015 to provide a flower petal type configuration. The radially extending slits may vary in number, depth, angle, and may result in straight or curved edges.

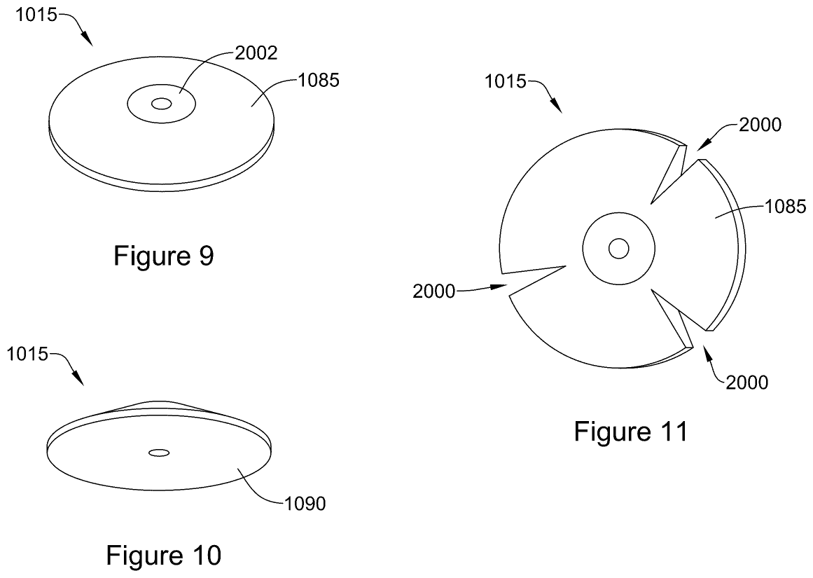

FIGS. 9 and 10 show another embodiment of the gasket 1015 that has a central, flat region 2002 surrounded by a convex top surface 1085. The bottom surface 1090 is flat. FIG. 11 shows another embodiment with one or more radially extending slits 2000 positioned on the gasket 1015 wherein the slits 2000 are smaller in size than those of the embodiment of FIG. 8.

As mentioned, one or more of the components of the vascular closure device 1000 can be manufactured of a bioresorbable material. Some examples are Purasorb PLC 7015 by CORBION; Resomer X 206 S, Resomer GC 753 S, or Resomer GT 643 S by EVONIK, and polymer blends comprised of lactide, trimethylene carbonate, & caprolactone. The material can vary.

A variety of sealant materials are within the scope of this disclosure. Some non-limiting examples include Porcine Gelatin (or other forms of gelatin), Oxidized Regenerated Cellulose, Polyurethane, Polyethylene Glycol (PEG), Fibrin (+ Thrombin), Sodium Carboxymethyl Cellulose, and collagen.

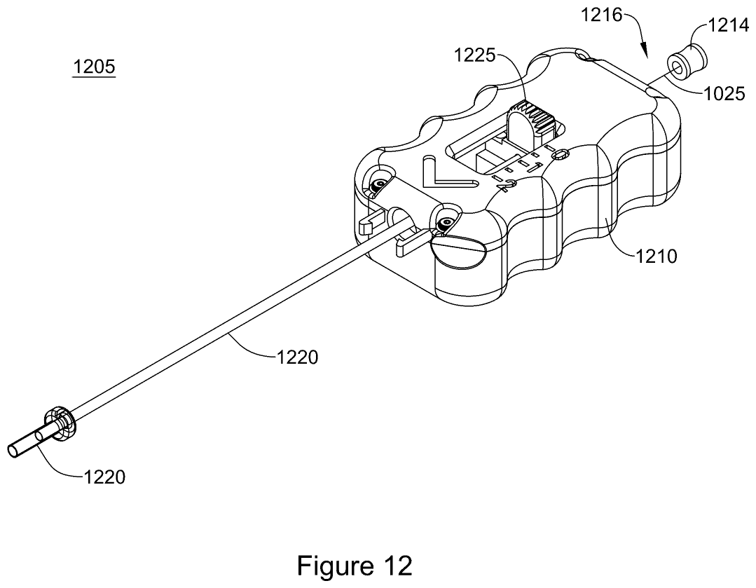

FIG. 12 shows a perspective view of a delivery device 1205 configured to deliver and deploy the vascular closure device 1000 to an arteriotomy of a blood vessel. As described further below, the delivery device 1205 is configured to introduce the vascular closure device 1000 into a blood vessel access sheath and deploy the vascular closure device 1000 at the arteriotomy. The delivery device 1205 locks onto the blood vessel access sheath and pushes the footplate 1005 into the blood vessel. The delivery device 1205 also sets the footplate 1005 in position against the vessel wall and then delivers the extravascular components (sealant, gasket 1015, and securement) into position for sealing the arteriotomy.

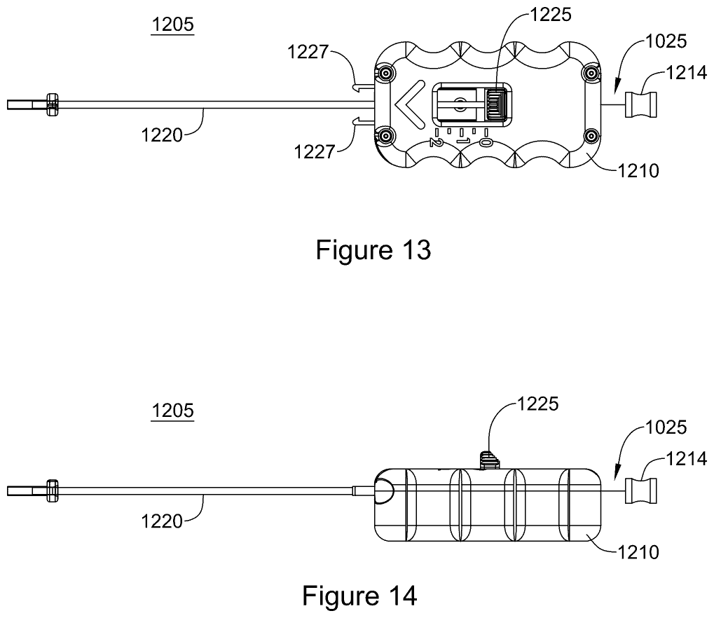

FIG. 13 shows a top view of the delivery device 1205 and FIG. 14 shows a side view of the delivery device 1205. The delivery device 1205 includes a proximal handle 1210 attached to a distally extending hollow carrier tube 1220. The carrier tube 1220 has an internal region such as a cavity that can at least partially contain the vascular closure device 1000. The handle 1210 has at least one actuator, such as a pusher 1225, that can be actuated to deployment of the vascular closure device 1000, as described further below. One or more attachment structures, such as prongs 1227, are configured to mate with a blood vessel sheath to securely attach such a sheath to the handle 1210 during use.

The tether 1025 extends distally out of the handle from the hollow carrier tube 1220. A distal end of the tether 1025 is attached to a tether holder 1214. The tether holder 1214 is a structure that can be grabbed and manipulated by a user for controlling the tether 1025 such as by pulling on it. The tether 1025 can form a slack distance 1216 between a proximal end of the housing 1210 and a distal end of the tether 1214.

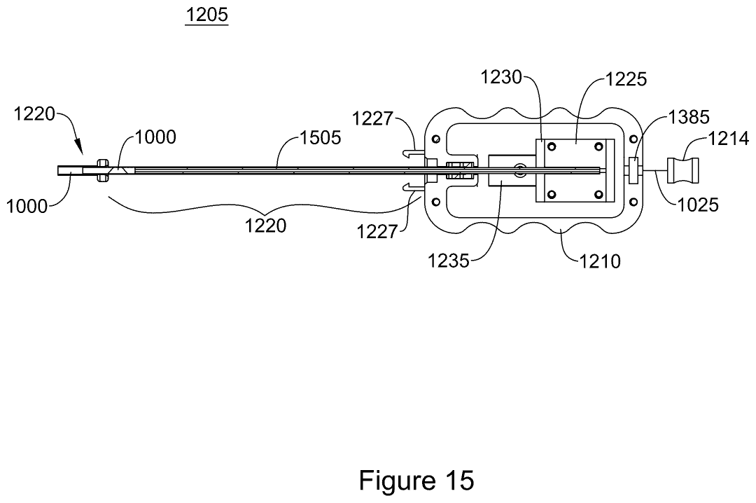

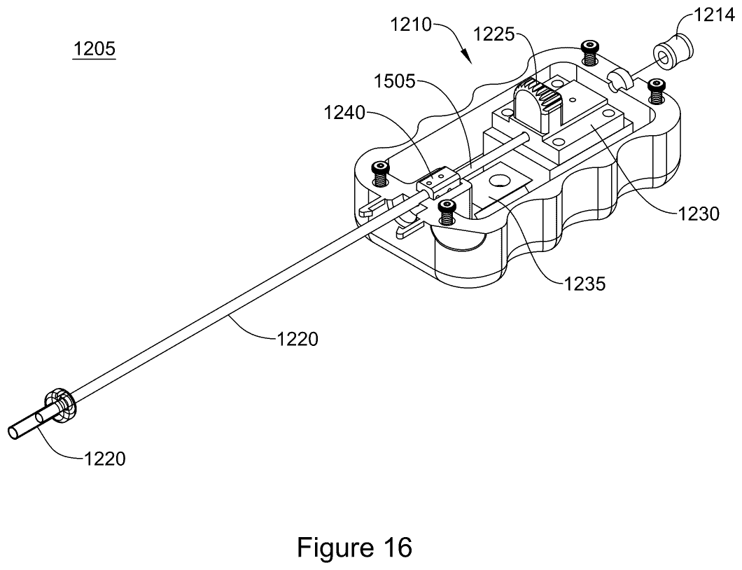

FIG. 15 shows a plan view of the delivery device 1205 showing internal components of the delivery device. FIG. 16 shows the delivery device 1205 with a cover of the handle 1210 removed. An elongated pusher tube 1505 is attached to the pusher 1225. The pusher tube 1505 movably extends through the carrier tube 1220. A distal tip of the pusher tube 1505 is at a distal region of the carrier tube 1220 near the carrier tube 1220 so that it can push a vascular closure device 1000 out of the carrier tube 1220 during use, as described more fully below. The tether 1025 extends through a bushing 1385 in the handle 1210.

With reference still to FIGS. 15 and 16, the pusher 1225 is attached to a slide 1230 that slides along a rail 1235 thereby allowing the pusher 1225 to move along the rail 1235 (such as along a direction that is aligned with a long axis of the carrier tube 1220) and control sliding movement of the pusher tube 1505.

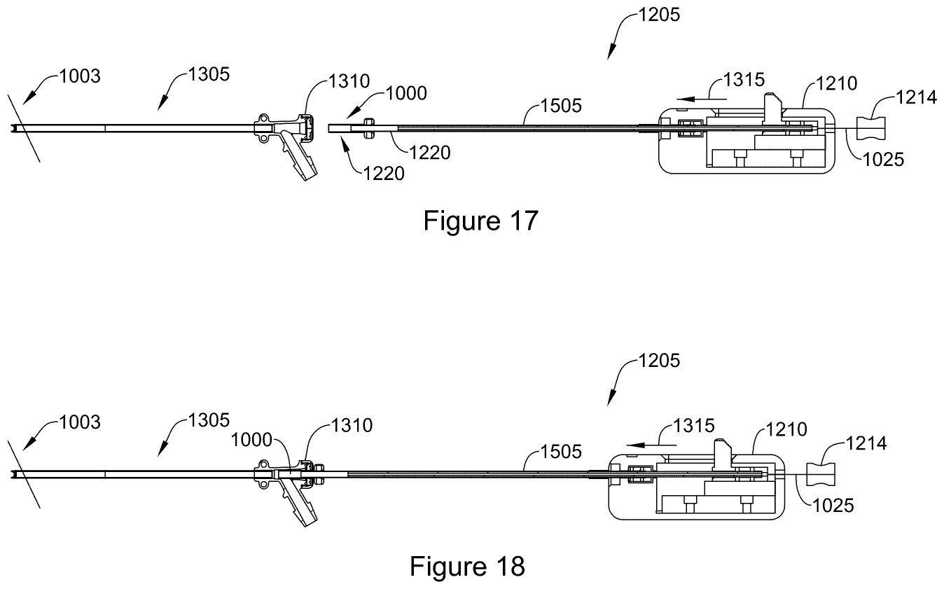

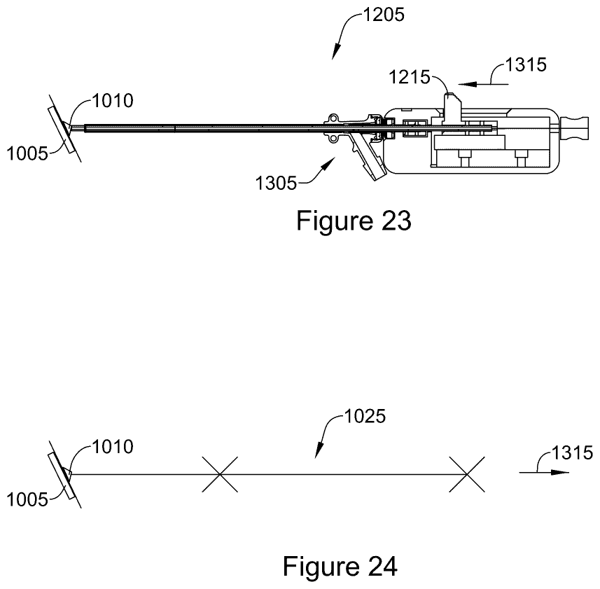

A method of use of the delivery device 1205 for delivering the vascular closure device 1000 is now described with reference to FIGS. 17-24. With reference to FIG. 17, the delivery device 1205 is positioned near an arterial access sheath 1305 that has been inserted into a blood vessel 1003 such as an artery, which can be for example a common carotid artery or any artery or any vein. The arterial access sheath 1305 has a distal end positioned inside the blood vessel 1003 via an arteriotomy. A hemostasis valve 1310 is at a proximal end of the arterial access sheath 1305. The carrier tube 1220 of the delivery device 1205 is positioned to align with the hemostasis valve and the entire delivery device 1205 is then slid forward toward the sheath 1305 as exhibited by arrow 1315. Prior to this, the vascular closure device 1000 in a collapsed state has been loaded into the distal region of the carrier tube 1220. As shown in FIG. 18, the distal region of the delivery device 1205 is next slidably inserted into the arterial access sheath 1305 as exhibited by arrow 1315.

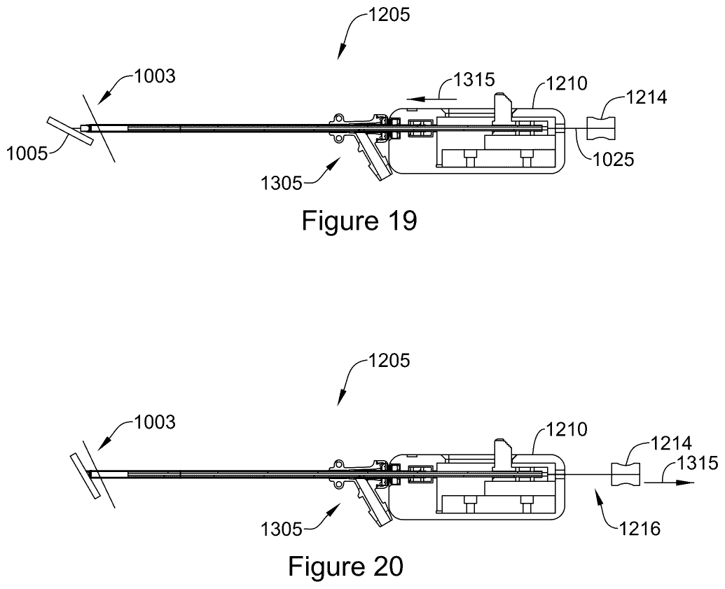

With reference to FIG. 19, the handle 1210 is advanced further toward the arterial access sheath 1305 (as exhibited by arrow 1315) until the footplate 1005 of the vascular closure device 1000 is pushed out into the blood vessel 1003. The footplate 1005 at this stage is hanging inside the vessel in an essentially floppy or unrestrained state. The footplate 1005 is not locked or restrained against the interior vessel wall.

As shown in FIG. 20, the tether holder 1214 is used to pull back (as exhibited by arrow 1315) the tether 1025 while the remainder of the delivery device remains stationary relative to the blood vessel wall. As discussed, the tether 1025 is attached to the footplate 1005 such that pulling back on the tether 1025 also pulls back the footplate 1005 toward the blood vessel inner wall. The friction bushing 1385 in the handle 1210 can hold the tether in place even when the tether holder 1217 is released by a user. The tether holder 1217 is pulled back to urge the footplate 1005 against the interior wall of the vessel and set the angle of the footplate 1005 against the distal tip of the arterial access sheath 1305. The vascular closure device 1000 is now locked on the arteriotomy of the vessel.

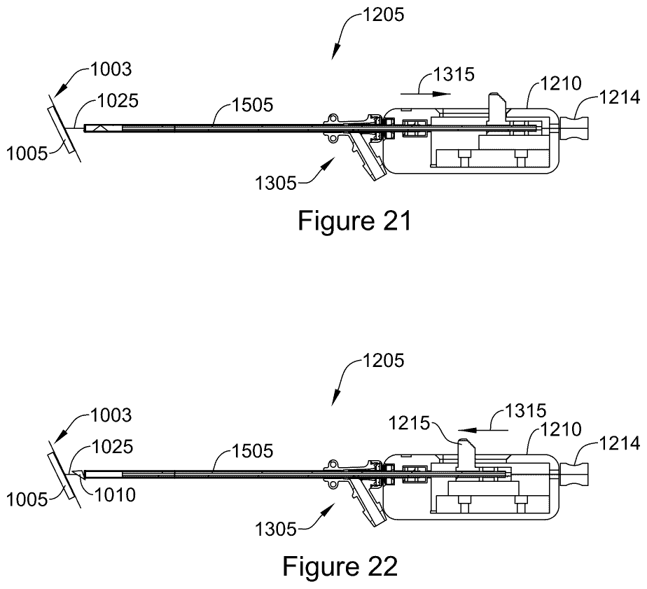

As shown in FIG. 21, the delivery handle 1210 is then pulled back (as exhibited by arrow 1315), which also pulls back the arterial sheath 1305 while the footplate 1005 remains locked on the arteriotomy of the vessel. This causes the footplate 1005 to gain apposition to the interior wall of the blood vessel 1003. The slack distance 1216 (FIG. 20) of the tether 1025 sets the amount of distance that the delivery device 1205 can be pulled back. With reference now to FIG. 22, the pusher 1215 is advanced (as exhibited by arrow 1315) to also advance the pusher tube 1505. A distal end of the pusher tube 1505 pushes the extravascular seal 1010 toward the blood vessel 1003 to expose the extravascular seal 1010 out of the distal end of the delivery device 1205.

With reference to FIG. 22, the pusher 1215 is then further advanced (as exhibited by arrow 1315) so that its distal end of the pusher tube 1505 compresses the extravascular seal 1010 against the footplate 1005 and seals the arteriotomy therebetween. The delivery device 1205 is then removed by cutting the tether at either side of the delivery device as exhibited by locations X as shown in FIG. 24. The first cut occurs between the tether holder, and the second cut occurs just beneath the patient's skin line such that no portion of the tether is outside of the patient.

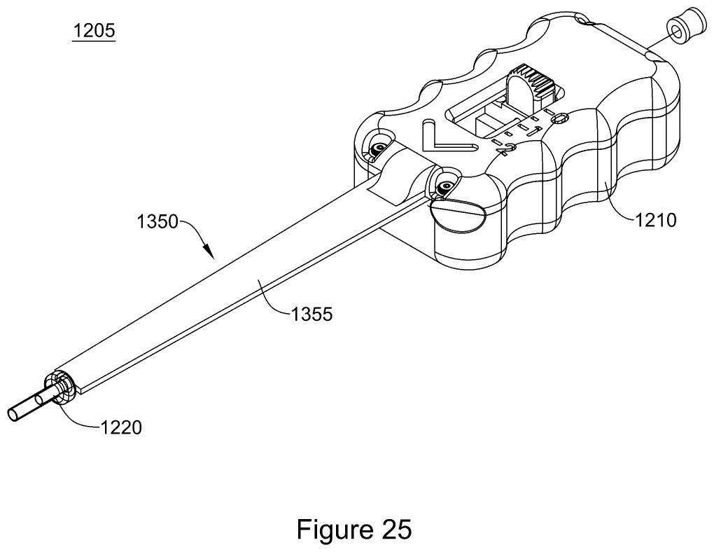

FIG. 25 shows the delivery device 1205 with a stabilizer 1350 coupled to the delivery device 1205. The stabilizer 1350 is a structure that a user can grasp onto to stabilizes the position of the delivery device 1205 during the initial use steps. The stabilizer can be removed from the delivery device 1205 once the delivery device 1205 is coupled to the arterial access sheath. The stabilizer 1350 is an elongated structure having a length that generally conforms to the length of the carrier tube 1220. A proximal end of the stabilizer 1350 can be coupled to the handle 1210 such that a distal end of the stabilizer 1350 is at or near a distal end of the carrier tube 1220. The stabilizer 1350 defines a planar surface 1355.

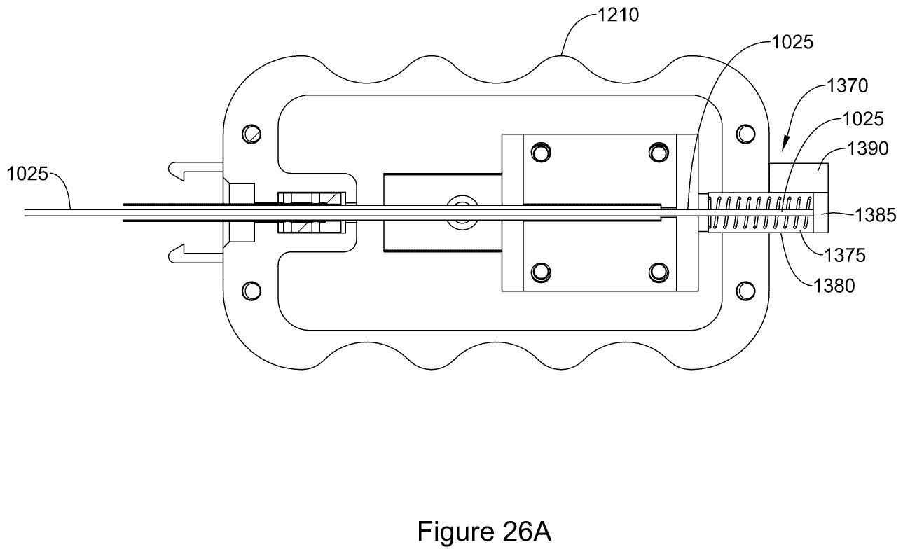

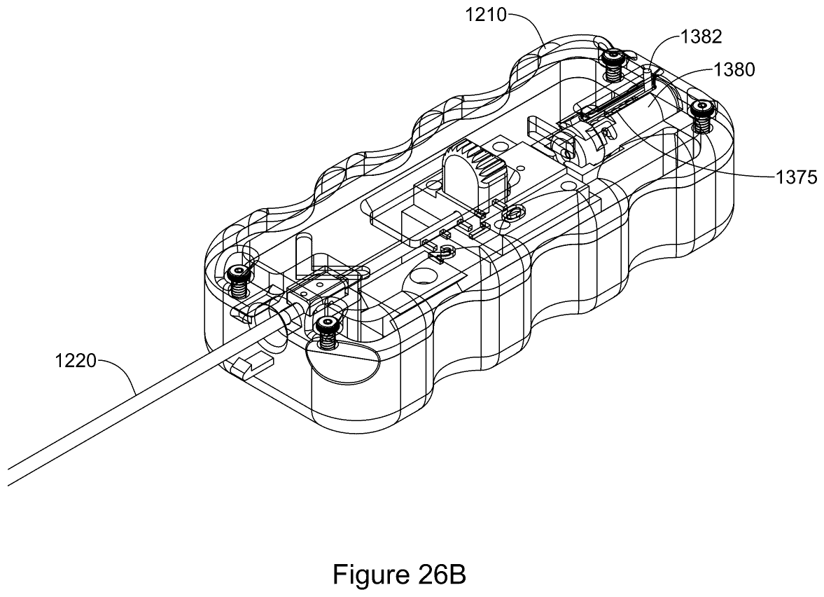

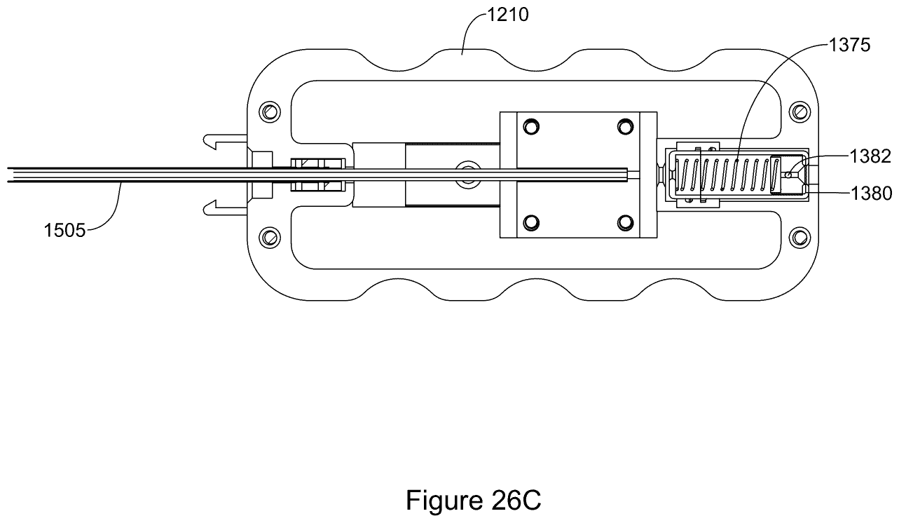

FIG. 26A shows a schematic representation of a spring-loaded tether holder assembly 1370 coupled to the handle 1210. The tether holder assembly 1370 is configured to maintain the tether 1025 in tension as the delivery device 1205 deploys the vascular closure device 1000. It removes the need for a user to manually manage the tether 1025 during use of the delivery device 1205. The tether holder assembly 1370 has a compression spring 1375 of relatively low spring force positioned in a housing 1380 and/or positioned in the handle 1210. The compression spring 1375 is attached to the tether 1025 (such as via a tether bushing 1385) to maintain tension on the tether 1025. The housing 1380 contains the bushing 1385, which is attached to a proximal end of the tether 1025. The bushing 1385 slides within the housing 1380 such that its position relative to an indication scale 1390 provides a user with an indication as to a level of tension force the spring 1275 exerts onto the tether 1025. The indication scale 1390 can include various indicators such as numbers, symbols, or other indicators that indicate tension force based on the position of an indicator arrow on the bushing 1385. In an embodiment, the indication scale is color coded with certain colors corresponding to an acceptable or desired range of tension force that corresponds to a step of the delivery method. This can reduce occurrences of exerting too much force, such as during the wall apposition step. It can also eliminate the need for user to manually manage the tether during deployment and compression.

FIGS. 26B and 26C shows an embodiment of the tether holder assembly 1370 that is positioned inside the handle 1210. The spring 1375 is mounted in the housing 1380 which is contained entirely in the handle 1210. A pin 1382 is attached to the spring 1375 and extends upwardly through a slot in the housing 1380. The position of the pin 1382 along the slot provides an indication of an amount of tensile force being applied to the tether. The pin 1382 protrudes from the spring housing 1380 to an outer surface of the handle 1210. The outer surface of the handle 1210 can include markings (such as numbers, letters, color, etc) that indicate, based on relative position to the protruding pin 1382, whether the level of tension is within an acceptable range for a particular step of delivery. For example, a first indicator can indicate that the force is acceptable for a wall apposition step while a second indicator can indicate that the force is acceptable for a vascular closure device compression step.

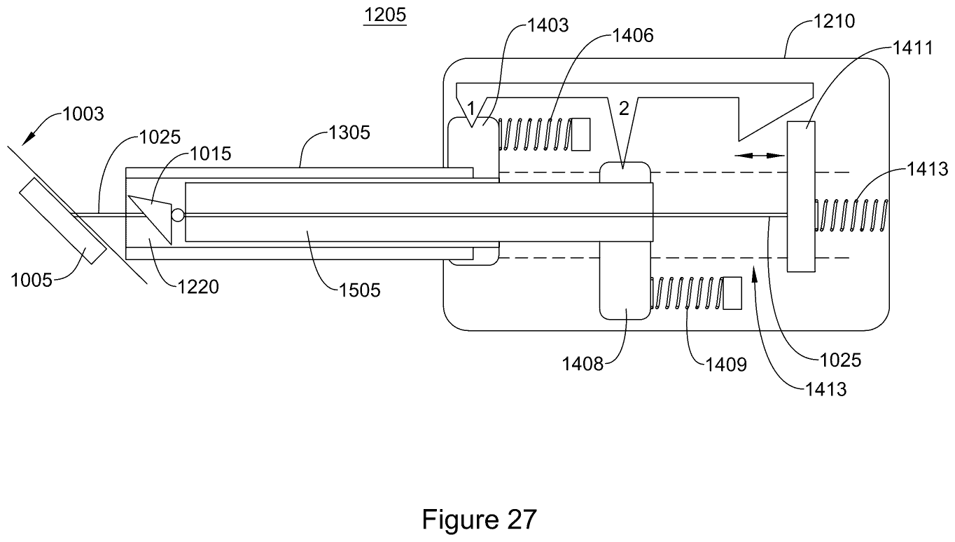

FIG. 27 shows a schematic representation of another embodiment of the delivery device 1205. This embodiment is configured to automatically pull back the arterial access sheath 1305 and automatically deploy and compresses the gasket 1015 against the arteriotomy of the blood vessel 1003. A deployment trigger comprises a tension force that ensures that the vascular closure device 1000 achieves vessel apposition.

With reference to FIG. 27 (which is not to scale), a proximal end of the sheath 1305 is attached to a sheath pull guide 1403 that is configured to slidably pull or move the sheath 1305 such as in a retraction direction (rightward in FIG. 27.) The sheath pull guide 1403 is coupled to a sheath spring 1406 which is in extension in FIG. 27 such that it pulls the sheath 1305 in the retraction direction when released. A proximal end of the pusher tube 1505 is attached to a pusher tube push guide 1408 that is configured to slidably push or move the pusher tube 1305 such as in a pushing or extension direction (leftward in FIG. 27.) The pusher tube push guide 1408 is coupled to a pusher tube spring 1409 which is in compression in FIG. 27 such that it pushes the pusher tube 1305 in the extension direction when released.

The tether 1025 is attached at a proximal end to a tether anchor 1411. The tether anchor 1411 is coupled to a tether spring 1413 which is in extension in FIG. 27 such that it maintains the tether 1025 in tension. One or more guide rails 1413 can be located in the handle 1210 for guiding movement of the sheath pull guide 1403, the pusher tube push guide 1408, and the tether anchor 1411. Also, a trigger release structure 1415 is coupled to the sheath pull guide 1403 and the pusher tube push guide 1408 to maintain those guides in place and to also release those guides upon interaction with the tether anchor 1411, as described below.

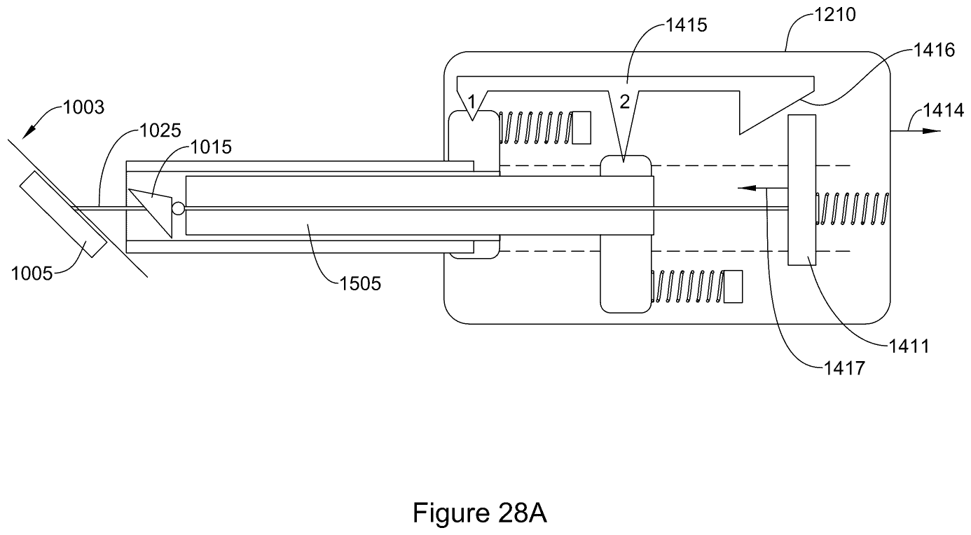

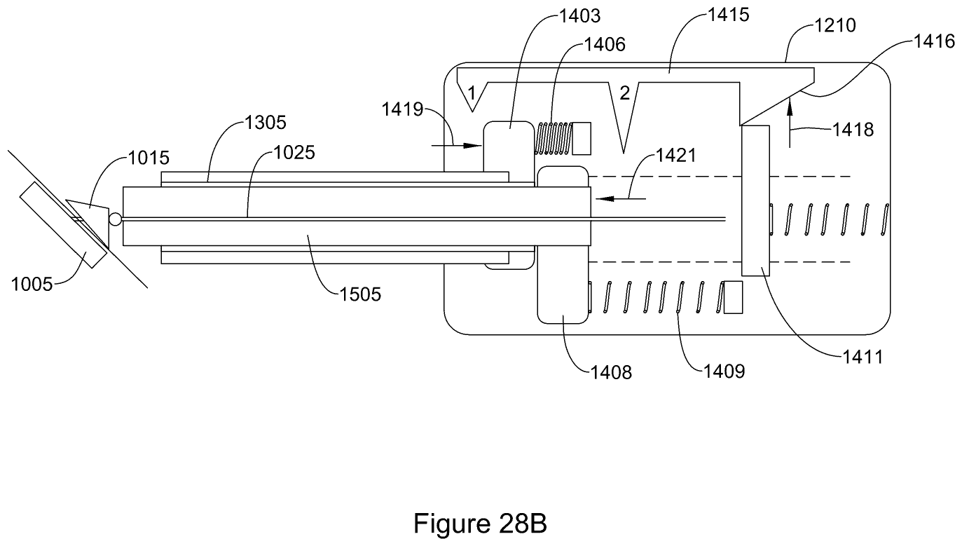

Pursuant to the method of use of the delivery device 1205 (as described above with reference to FIGS. 17-24), during the method the handle 1210 is pulled back (as represented by arrow 1414 in FIG. 28A) relative to the blood vessel 1003. With reference to FIG. 28A, this causes the suture anchor 1411 to slide on the guide rails 1413 toward a ramped portion 1416 of the trigger release structure 1415, as represented by arrow 1417.

With reference now to FIG. 28B, the suture anchor 1411 eventually abuts the ramped portion 1416 of the trigger release structure 1415 and pushes the trigger release structure 1415 upward (as represented by arrow 1418) into a release position. In the release position, the trigger release structure 1415 disengages from both the sheath pull guide 1403 and the pusher tube push guide 1408. The sheath spring 1406 then pulls and retracts the sheath 1505 (as represented by arrow 1419) away from the blood vessel 1003. The pusher tube spring 1409 pushes and extends the pusher tube 1505 (as represented by arrow 1421) toward the vascular closure device 1000. The distal end of the pusher tube 1505 pushes the vascular closure device's extravascular portion (e.g., the gasket 1015) against the blood vessel wall to achieve vessel apposition by the footplate 1005.

The trigger release structure 1415 can alternately be replaced or supplemented with two manual triggers. A first trigger can manually activated to release the sheath pull guide 1403 and a second trigger can be manually activated to release the pusher tube push guide 1408.

### Additional Embodiments of Vascular Closure Device

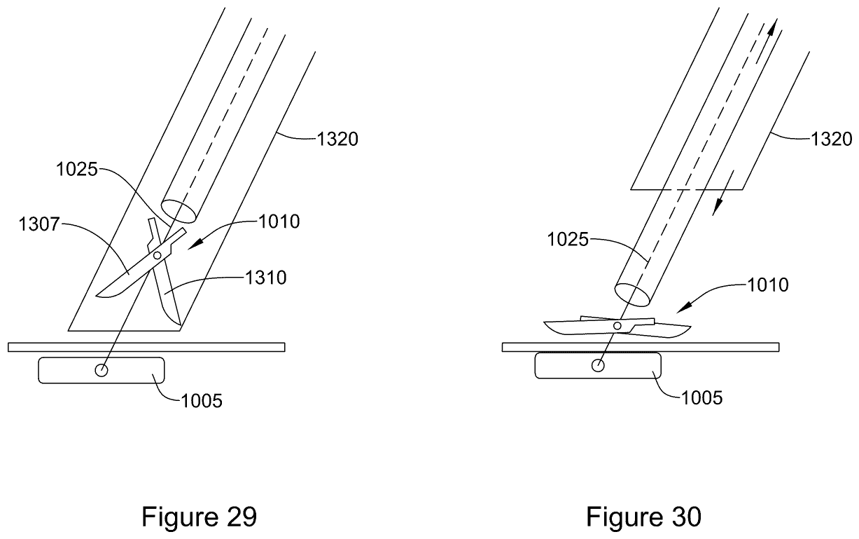

In an implementation shown in FIG. 29, the extravascular seal 1010 is configured such that is formed of two or more pieces 1305 and 1310 that are attached to one another (one on top of the other) and pivotably fastened together at hinge 1215. This permits the two pieces 205 and 210 to rotate relative to one another between a closed, contracted state (as shown in FIG. 29) and an open, expanded state (as shown in FIG. 30). When positioned inside a delivery sheath 1320 as shown in FIG. 2, the two pieces 1307 and 1310 form a closed state of overall reduced size that permits fitting within the sheath 1320. Once deployed distally outside a distal opening of the sheath 1320 as shown in FIG. 30, the two pieces 1307 and 1310 open or expanded relative to one another such as to anchor the closure device in place against tissue. A suture, such as the tether 1025, can be used to cinch the extravascular seal 1010 in place and lock it in position relative to the footplate 1005 and the blood vessel wall. Alternatively, the scissor configuration may lock on its own to a standard pressure, width, or distance from the footplate 1005 by using ratchets or other locking mechanisms known in the art.

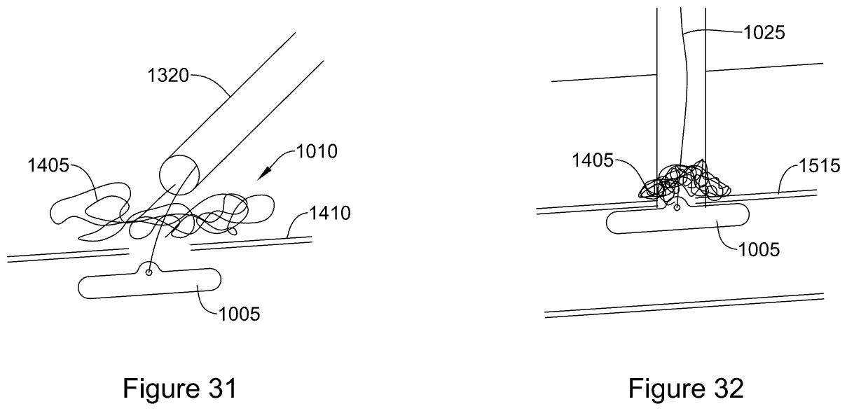

In another implementation shown in FIG. 31, the extravascular seal 1010 includes or incorporates an embolization coil 1405 that is configured to tether the extravascular seal 1010 to the vessel 1410 and seal the arteriotomy. The coil 1405 can be Nitinol or bioresorbable. A suture such as the tether 1025 anchors the coil 1405 to the intravascular footplate 1005 and against the exterior vessel wall (either as part of the coil or as a separate length of suture).

In an implementation shown in FIG. 32, the bioresorbable footplate 1005 is connected to the tether 1025. An extravascular bioresorbable sealant 1510 is coupled to the footplate 1005 once the footplate 1005 is positioned against the vessel intravascular wall 1515 and used to seal the arteriotomy. In use of this configuration, an “inverse” manual compression is used to hold the distal seal in place by clamping the tether at the patient's skin while the sealant is injected and while the sealant cures. This seals the arteriotomy and holds the intravascular footplate 1005 in place. The sealant can vary and can be comprised, for example, of PEG, bioresorbable adhesives (one part or two part), UV curable adhesives, hydrogels, clotting agents (such as collogen), or any other flowable and curable bioresorbable materials known to the art.

In another implementation, a glue or sealant is injected between the extravascular seal 1010 and the intravascular footplate 1005 to further seal the arteriotomy and hold the extravascular seal 1010 and the intravascular footplate 1005 in place.

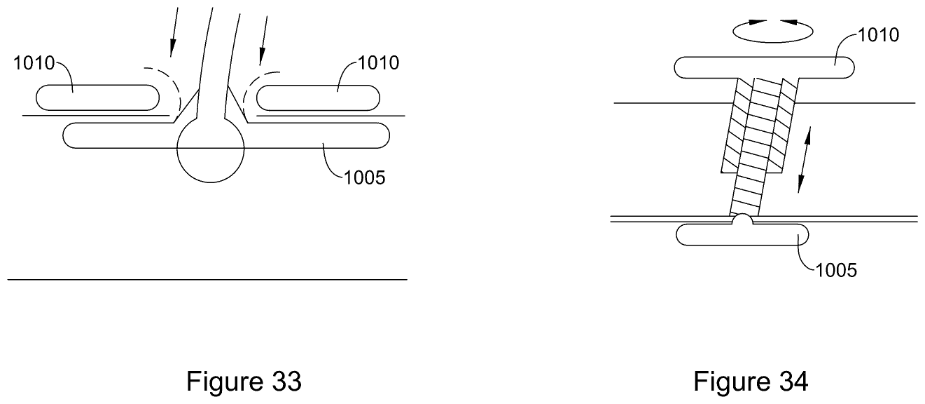

In another implementation shown in FIGS. 33 and 34, the extravascular seal 1010 and the intravascular footplate 1005 mechanically mate to one another (such as in direct contact) such as in a way that holds them together and seals the arteriotomy. For example, the extravascular seal 1010 and the intravascular footplate 1005 can mechanically snap to one another as shown in FIG. 33. FIG. 34 shows an embodiment where the extravascular seal 1010 and the intravascular footplate 1005 have co-axial portions that engage one another in a ratchet manner. Other mechanical couplings are possible including a screw, bond, lock, or other mechanical engagement that permits the extravascular seal 1010 and the intravascular footplate 1005 to mate to one another.

When the extravascular seal 1010 and the intravascular footplate 1005 mate or otherwise engage one another, a feel, sound, visual indicators, or a combination thereof can occur to provide feedback to the user that the device is in place. Additionally, the extravascular seal 1010 and the intravascular footplate 1005 can be configured to mate together in a manner that enables the user to control the force applied to the arteriotomy and vessel wall and/or the distance between the components (e.g. screw, torque, snap, ratchet, cinch, break, etc.). Any of implementations described herein may be combined to enhance or modify the seal of the arteriotomy to provide definitive closure. For example, an adhesive, such as a PEG adhesive, may be applied to the gasket 1015 to act as a “belt and suspenders” way of securely closing an access site. Other combinations are possible as well.

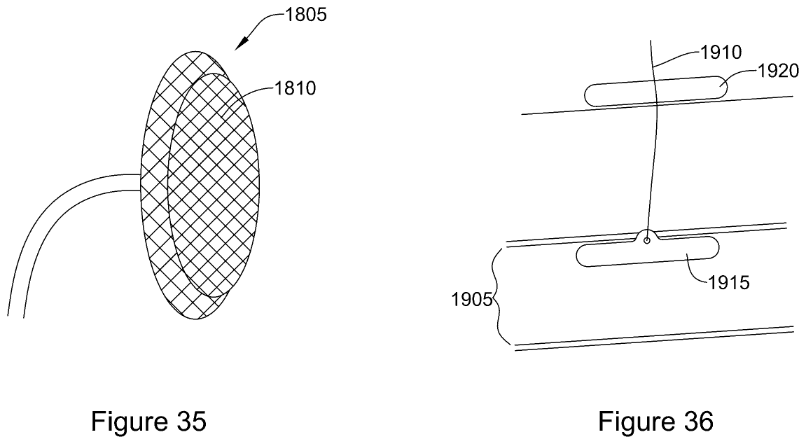

In another implementation shown in FIG. 35, the closure device is an occluder style closure device such as a septal occluder configured to plug or otherwise occlude an arteriotomy during a transcarotid procedure. In the example shown in FIG. 35, the occluder device 1805 includes an occluding assembly 1810 formed of one or more wire mesh frame (such as Nitinol) with a thin polyester fabric cover. The occluding assembly has an expanded, disc shape that occludes an arteriotomy. The fabric cover can vary in configuration and can be for example knit, woven, and other patterns.

Similarly, the intravascular footplate 1005 described above in prior implementations can be configured as an occluder disc (with or without Nitinol—i.e., a bioresorbable mesh gasket) and be combined with the extravascular gasket 1015. The extravascular gasket 1015 can resemble one of the occluder disc (or a sphere) and be combined with an intravascular footplate 1005.

There are now described example methods for percutaneous closure of the carotid arteries. Pursuant to an example method, a user accesses the lower pressure venous system of a patient and crosses over into the arterial system near a desired treatment site. For the application of carotid artery stenting, access may be gained via an access location in the peripheral venous system such as, for example, the femoral artery).

The user then tracks a guide sheath or catheter from the access location to the jugular vein, which is closely proximal to the carotid artery. Once the guide sheath is in the jugular vein, a true lumen re-entry device is used to cross into the arterial system (such as the carotid artery) and create a temporary fistula between the vein and the artery. Although the venous system contains valves, those valves can be crossed by those trained in interventional procedures. Additionally, the venous system typically has less disease than the arterial system and the venous system flow leads to the lungs, not the brain. Therefore, plaque is less likely to be disrupted when tracking to the treatment site and there is very little risk of causing a stroke. After crossing from the venous system to the arterial system, the treatment site can be stented, and the fistula can be closed using a closure device.

The arteriotomy such as in the carotid artery is thus closed via a vein such as the jugular vein. This provides some safety benefits in that closure leakage can be guided or drawn into the fistula/venous system, preventing blood loss. Additionally, closure of a venous access site (e.g., femoral, jugular, etc.) is known to be easier than closing an arterial access site due to the lower pressure. In another implementation, a user directly accesses the jugular vein via percutaneous means and the carotid artery treated as described.

A closure device may fail for any of a variety of reasons, such as due to a dysfunctional device, operator error, incompatibility with the anatomy, etc. In such failure situations, it can be advantageous to have a bailout solution in place, which can be a mechanism and/or method wherein the mechanism or method enables a user to stabilize the patient until a repair can be made.

Pursuant to an exemplary bailout solution, a tether (such as the tether 1025 of the vascular closure device 1000) is used for inverse manual compression in the region of the carotid artery. In peripheral blood vessel regions (such as the femoral artery), manual compression is a common method to stop or slow bleeding at the peripheral access sites. Pursuant to such manual compression, a user applies pressure to the access site by hand until the bleeding stops or until a surgical repair can be made. Although this is an effective strategy on peripheral blood vessels, manual compression can be difficult or ineffective on the carotid arteries. In contrast to peripheral vessels, the carotid artery is more mobile and does have an adjacent bone (such as directly underneath it) to provide support for compression from an external location. Moreover, the carotid arteries are adjacent to the patient's airway, which should not be compressed. A traditional manual compression method thus is not effective for the carotid vasculature.

An example bailout method is now described with reference to FIG. 36, which schematically shows a side view of an artery 1905 wherein a tether 1910 is attached to an intravascular footplate 1915 positioned in an artery adjacent an arteriotomy. The tether is also attached to a clamp member 1920 positioned proximal the footplate 1915 such as at a skin surface 1920. Pursuant to this method, the arteriotomy is plugged from the inside the artery rather than pushing the vessel closed from outside the artery (such as pursuant to traditional manual compression). Plugging the artery from the inside out avoids the issues discussed above relating to vessel mobility, support, and the airway.

Pursuant to an inverse manual compression procedure, a user pulls the tether 1910 up or away from the artery. This pulls the footplate 1915 upward toward the arteriotomy causing the footplate 1915 to plug the arteriotomy. This inverse manual compression stops or inhibits bleeding via the arteriotomy. The tether can be placed in tension at the level of the skin. This enables this bailout method to work without having to continuously hold the footplate 1915, which serves as an arteriotomy plug, in place. This process can be used with a variety of gasket-style closure devices while the tether reduces the risk of device embolization.

Pursuant to another bailout solution, an arterial sheath is re-inserted through the arteriotomy after initial removal wherein the reinsertion of the arterial sheath serves to plug the arteriotomy. This process can be used such as if a closure device is shown to be ineffective. A guidewire is placed through the arteriotomy and remains while the closure device is removed from the arteriotomy after initial placement.

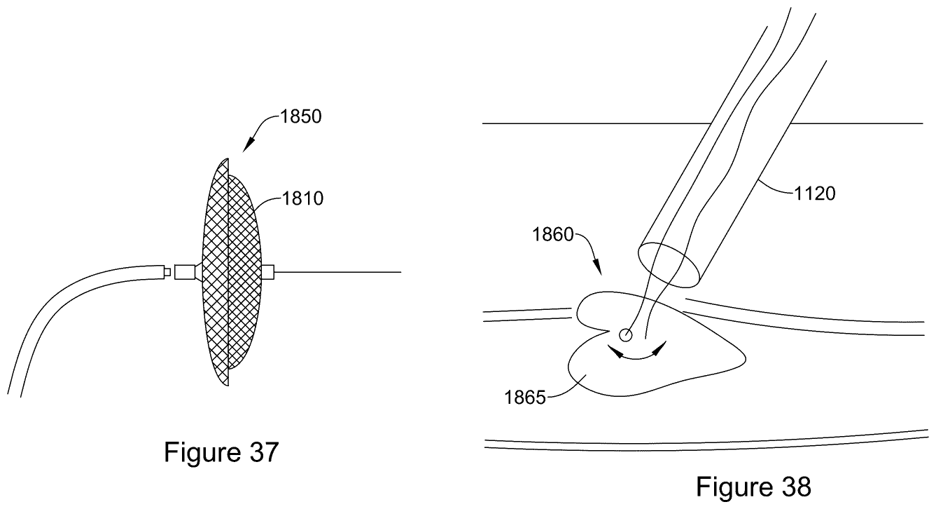

FIG. 37 shows an embodiment of an occluder style closure device 1850 that is configured to be removed or recaptured from the arteriotomy after initial deployment. For percutaneous carotid artery closure (PCAC), the closure device 1850 is scaled in size to fit a carotid arteriotomy created while ensuring guidewire positioning is maintained and the device can be recaptured after initial deployment to assess closure performance. The closure device 1850 may be constructed using Nitinol wire mesh frames with thin polyester fabric covers. The fabric cover can be radially expanded from a reduced size delivery state by twisting or shortening its length. A reverse motion (such as reverse twisting or lengthening) returns the closure device 1850 to its reduced-size delivery state such that it is removable from the arteriotomy.

FIG. 38 shows another embodiment of a closure device 1860 for a blood vessel 1861, the closure device 1860 formed of an inner closure member 1865 such as a footplate or a gasket. The inner closure member 1865 is shaped in a manner that enables the inner closure member to be pulled back into a distal opening of an arterial sheath 11120. In the illustrated embodiment, the inner closure member 1865 has a shape that forms into a tip region of reduce size wherein the tip region is sized and shaped to fit within distal opening of the arterial sheath 1120. For example, the inner closure member 1865 can be heart shaped or spade shaped. Upon positioning the tip region in the distal opening of the arterial sheath 1120, a tether or other mechanism can be used to pull the inner closure member 1865 into the distal opening of the arterial sheath 11120 thereby causing the larger end to fold up when pulled into the sheath. If there is a desire to recapture the inner closure member 1865 following initial deployment, the proximal gasket can be released. A push rod can then be used to move the inner closure member 1865 away from the intravascular vessel wall and pivot it so that the tip of the inner closure member 1865 is aligned towards the sheath, and then the entire closure member can be pulled through the sheath and removed. A guidewire can be left in place so that the arterial can be reinserted for closure device removal and to plug the arteriotomy.

One embodiment to enable a releasable proximal gasket utilizes a pawl and ratchet mechanism that allows the system to tightened and released as often as desired. Another embodiment to enable a recapturable system to maintain guidewire access is a “Buddy-Wire Passage” configuration where a geometry is created in the distal footplate and proximal gasket that allows a guidewire to remain in the vessel up until final locking/deployment of the closure device.

As discussed above, when a closure device fails, the traditional bailout method is to apply manual compression at the access site until either the bleeding stops or until a surgical repair can be performed. However, such manual compression is generally impractical in the carotid region. There is now disclosed percutaneous methods to limit the bleeding until a surgical repair can be made.

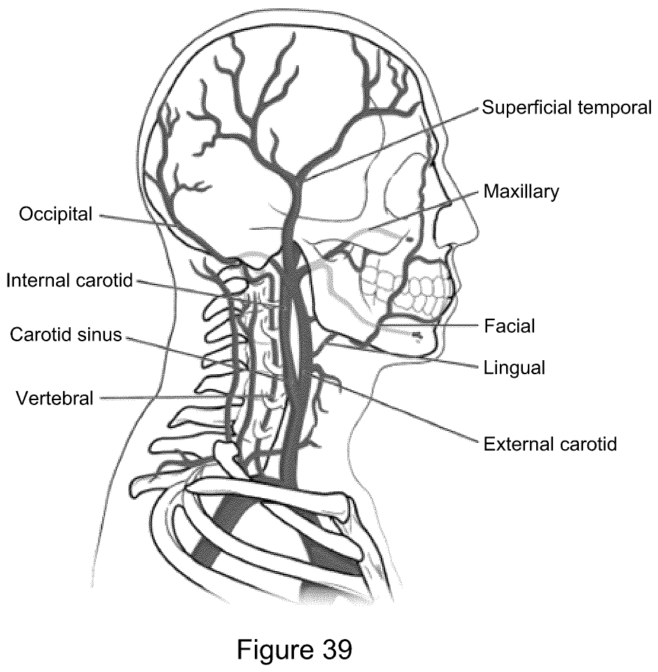

A transcarotid artery revascularization procedure includes accessing the common carotid artery (CCA) and stenting of the internal carotid artery (ICA). FIG. 39 shows a schematic representation of the vasculature in the neck and skull region. The ICA traverses deep into the patient's skull and carries blood to the patient's brain. A vascular closure device, which leaks, is placed at the CCA, which can be conducive to the following methods when a vascular surgeon is not available to make a surgical repair. In one example method, at least one highly compliant balloon is delivered percutaneously and intravascularly to the CCA access site via a pathway that includes one or more vessels other than the CCA. The balloon is inflated from inside the vessel to plug the hole and stop bleeding. A single balloon can be used to plug the arteriotomy site directly or a multi-balloon system can be used to occlude the CCA proximal and distal to the arteriotomy.

In an alternate method, the external carotid artery (ECA) is percutaneously utilized to deploy the balloon. One or more access sites include the standard peripheral access vessels (e.g., femoral, radial, axial, etc.) or the ECA. The ECA delivers blood to the face and skull and many of its branches remain superficial, including the superficial temporal artery. This artery could be accessed quickly and provide a relatively straight path to the CCA, making delivery fast and easy. Additionally, if a guidewire is maintained during initial deployment of the closure device and if the closure device is retrievable, then highly compliant balloons can also be delivered through the arteriotomy.

In an embodiment, the external carotid artery (ECA) is accessed via a branch of the ECA such as the superficial temporal artery or the occipital artery (or other branch of the ECA) for example. A closure device is then delivered to an arteriotomy of the common carotid artery via a retrograde direction from the ECA. Retrograde delivery of the closure device allows for better control and opportunity for bailout such as via a 3-4F access device. The closure device can be a balloon. For example, two or more balloons are positioned in the common carotid artery wherein the arteriotomy is positioned in a space between at least a first balloon and second balloon. For example, a distal balloon and a proximal balloon are positioned such that the arteriotomy is positioned in a space between the distal balloon and the proximal balloon. In another embodiment, the balloon is elongated along a length L and positioned such that the arteriotomy lies along the length L of the balloon.

There are now disclosed systems and methods configured to monitor patient bleeding following percutaneous closures of the carotid arteries. After closing an access site in the carotid arteries, monitoring of bleeding at the access site can be very important. If bleeding occurs and goes unchecked, the patient airways can become compromised due to tissue swelling, which can lead to complications including death. If properly detected, a practitioner can take action alleviate the leak and protect the patient's airway.

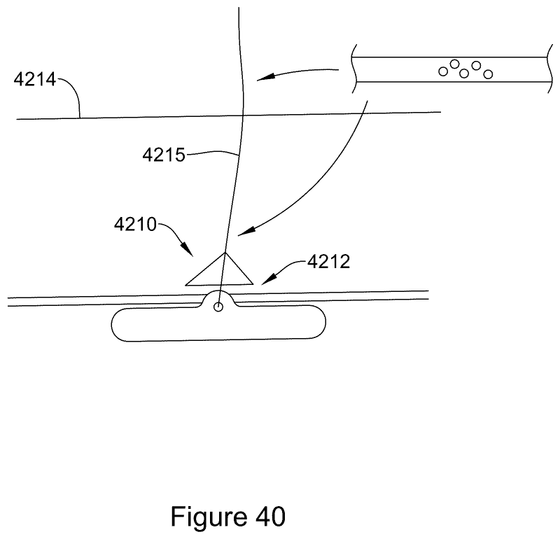

With reference to FIG. 40, a closure device 4210 is positioned at the arteriotomy 4212 with a tether 4215 attached to the closure device 4210. The tether 4215 can be for example a hollow fiber that includes one or more porous sections with irrigation holes that extend from the closure device 4210 to the skin surface 4214. The irrigation holes provide a mechanism for a user to ascertain that blood has leaked from the artery, which can be observed above the skin.

In another embodiment schematically shown in FIG. 41, a wick or a capillary tube extends from the closure device at the arteriotomy to the skin. Blood passes through the wick to a portion of the wick that is visible at the skin's surface. The wick may also act as a tether or be intertwined with or incorporated into the tether. A mid portion of the wick may be coated or covered to reduce blood absorption from the subcutaneous tissue but remain open (or absorbent) at the arteriotomy.

In another embodiment, the closure device includes or is coupled to a “detachable” bleed-back indicator wherein the bleed-back indicator comprises a small tube, a capillary tube (or meniscus), an irrigation tube, or other conduit that detaches from the closure device during closure and remains in the tissue track for patient monitoring. During deployment of the closure device, the bleed-back indicator assists with device positioning within the vessel and after closure, it is left behind with one end at the closure site and the other end at the surface of the vessel to provide an indication of continued bleeding.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore, the spirit and scope of the appended claims should not be limited to the description of the embodiments contained herein.

1. An arterial closure device, comprising:
a proximal gasket configured to be positioned on a first side of a blood vessel wall;
a distal footplate configured to be positioned on a second side of the blood vessel wall opposite the proximal gasket;
a seal configured to be positioned on the first side of the blood vessel wall between the proximal gasket and the blood vessel wall;
a tether coupled to at least the proximal gasket and the distal footplate; and
a securement element on the tether, the securement element configured to secure the proximal gasket and the seal against the first side of the blood vessel wall.

2. The arterial closure device of claim 1, wherein the upper gasket is rigid.

3. The arterial closure device of claim 1, wherein the upper gasket is flexible.

4. The arterial closure device of claim 1, wherein the upper gasket and the seal are attached to one another.

5. The arterial closure device of claim 1, wherein the seal is configured to conform to a shape of the blood vessel wall.

6. The arterial closure device of claim 1, wherein the proximal gasket, the distal footplate, and the seal are bioresorbable.

7. The arterial closure device of claim 6, wherein the distal footplate bioabsorbs prior to the proximal gasket and the seal.

8. The arterial closure device of claim 6, wherein the distal footplate bioabsorbs in approximately 30 days.

9. The arterial closure device of claim 1, wherein the distal footplate includes a stiff core surrounded by one or more flexible wings.

10. The arterial closure device of claim 9, wherein the distal footplate includes at least one raised surface configured to form a sealing relationship with the second side of the blood vessel wall.

11. The arterial closure device of claim 10, wherein the distal footplate includes one or more tether holes sized and shaped to receive the tether to secure the tether to the distal footplate.

12. The arterial closure device of claim 11, wherein the one or more tether holes are arranged in a diamond pattern, wherein the diamond pattern has a long axis that aligns with a long axis of the stiff core.

13. An arterial closure device, comprising:
an intravascular footplate configured to be disposed within a blood vessel;
an outer gasket configured to be disposed adjacent to an exterior wall of the blood vessel;
a seal configured to be disposed between the exterior wall of the blood vessel and the outer gasket;
a tether coupled intravascular footplate and the outer gasket; and
a securement member coupled to the tether, the securement member being configured to secure the outer gasket and the seal against the exterior wall of the blood vessel.

14. The arterial closure device of claim 13, wherein the seal is configured to conform to the exterior wall of the blood vessel.

15. The arterial closure device of claim 13, wherein one or more of the intravascular footplate, the outer gasket, and the seal are bioresorbable.

16. The arterial closure device of claim 13, wherein the intravascular footplate includes a core surrounded by one or more wings.

17. The arterial closure device of claim 13, wherein the intravascular footplate includes a raised surface configured to form a sealing relationship with an interior wall of the blood vessel.

18. The arterial closure device of claim 13, wherein the intravascular footplate includes one or more tether holes configured to receive the tether.

19. The arterial closure device of claim 18, wherein the one or more tether holes are arranged in a pattern.

20. An arterial closure device, comprising:
an intravascular footplate configured to be disposed within a blood vessel and configured to seal against an inner wall surface of the blood vessel;
an extravascular seal configured to be disposed along an exterior wall of the blood vessel;
an outer gasket configured to be disposed along an outer surface of the extravascular seal;
a tether coupled intravascular footplate and the outer gasket; and
a securement member coupled to the tether, the securement member being configured to secure the outer gasket and the extravascular seal against the exterior wall of the blood vessel.
